(19) United States
(12) Patent Application Publication
(10) Pub. No.: US 2019/0027253 A1
(43) Pub. Date: Jan. 24, 2019

(54) PRECISION HEALTH INSIGHT TOOL

(71) Applicant: Accenture Global Solutions Limited, Dublin (IE)

(72) Inventors: James Hendry Hoover, Mechanicsburg, PA (US); Katherine Kramer, Leesburg, VA (US); Oliva Marie Casto, Washington, DC (US); Donnalee Sykes, Reston, VA (US); Jeffrey Plummer, Annandale, VA (US); Timothy Krappweis, Herndon, VA (US); William Moran, Fairfax, VA (US); Kimberly Green, Arlington, VA (US); Christopher David, Leesburg, VA (US); Sarah Krappweis, Fairfax, VA (US); Christopher Stewart, Ashburn, VA (US); Jeffrey Morris, Ashburn, VA (US); Dwayne Alan Nelson, South Boston, VA (US); James Neighbors, Burke, VA (US); Gi Yeun Kang, Centreville, VA (US); Darsh Nand, Bethesda, MD (US); Patty Horoho, Clifton, VA (US); Denise Hopkins-Chadwick, Cibolo, TX (US)

(21) Appl. No.: 16/022,209

(22) Filed: Jun. 28, 2018

Related U.S. Application Data
(60) Provisional application No. 62/536,274, filed on Jul. 24, 2017.

(51) Int. Cl. G16H 50/30; G06Q 50/26

(52) U.S. Cl. CPC G16H 50/30; G16H 50/20; G06Q 50/26

(57) ABSTRACT

Methods, systems, and apparatus, including computer programs encoded on a computer storage medium, for determining a likelihood that an individual is ready. The methods, systems, and apparatus include actions of obtaining health data for a set of individuals, providing the health data for the set of individuals to a readiness classifier that classifies individuals as either ready or as not ready, obtaining, from the readiness classifier, classifications for each of the individuals of the set of individuals, generating, from the classifications for each of the individuals of the first set of individuals, a readiness risk scoring model that determines a risk that an individual is not ready from health data of the individual, obtaining health data regarding a particular individual, and determining, from the health data regarding the particular individual and the readiness risk scoring model, a risk that the particular individual is not ready.

### CROSS-REFERENCE TO RELATED APPLICATIONS

This application claims the benefit of U.S. Provisional Patent Application No. 62/536,274, filed Jul. 24, 2017, which is hereby incorporated by reference in its entirety.

### TECHNICAL FIELD

This disclosure generally relates to health monitoring.

### SUMMARY

In general, an aspect of the subject matter described in this specification may involve a process for determining a likelihood that an individual is ready for deployment.

Withdrawing an individual from deployment may be costly, unsafe, and cause delay. For example, an individual may get sick or become injured during a deployment and may need to be medically evacuated before the mission is completed, and another individual may need to be deployed as a substitute. Additionally, determining that an individual is not ready for deployment during a health screen right before the deployment may be costly, unsafe, or cause delay as a substitute may need to be found at short notice.

To avoid some issues, a system may use pre-set policies to determine whether an individual is ready for deployment and only deploy individuals that are ready. For example, a system may apply a pre-set policy that individuals with a body mass index (BMI) greater than thirty should be classified as not ready to be deployed and only individuals classified as ready to be deployed may be deployed. However, some individuals may have a last recorded BMI of less than thirty but then right before deployment be tested to have a BMI greater than thirty so need to be replaced, or may be rapidly increasing in BMI before deployment and in the middle of deployment increase to a BMI of thirty four and get sick. Accordingly, pre-set policies may be insufficient.

Accordingly, a system may use historical data regarding individuals along with policies to determine a likelihood that an individual is not ready. For example, the system may identify individuals that had to return early from deployment for health reasons so actually were not ready, and then use characteristics of those individuals before they were deployed to identify characteristics that may indicate that individuals are not ready for deployment. The system may generate a readiness risk scoring model from the identified characteristics, and then use that readiness risk scoring model to determine a risk that other individuals are not ready for deployment. The risk that individuals are not ready for deployment may inversely correspond to a likelihood that the individuals are ready for deployment.

The system may be provided in the form of a tool that individuals may use to see their own likelihood of readiness and how they might improve the likelihood that the system calculates. Individuals may also view likelihoods of other individuals to identify whether sufficient individuals are ready for deployment, and whether any actions should be taken on an individual or group level to increase likelihood of readiness.

Implementations of the present disclosure may provide one or more of the following advantages. Cost, safety, and timeliness issues may be reduced by identifying individuals that satisfy readiness requirements but have a high likelihood of not being ready. For example, individuals that currently satisfy readiness requirements but have a high likelihood of not being ready, e.g., individuals with rapidly increasing BMI that currently just barely satisfy BMI requirements, may be identified and not considered ready. In addition, warning and suggestions provided by the system may result in an increase in overall likelihood of readiness of individuals. For example, individuals may be warned that a likelihood of readiness for themselves or other individuals is reducing and, in response, take corrective action.

In some aspects, the subject matter described in this specification may be embodied in methods that may include the actions of obtaining health data for a set of individuals, providing the health data for the set of individuals to a readiness classifier that classifies individuals as either ready or as not ready, obtaining, from the readiness classifier, classifications for each of the individuals of the set of individuals, generating, from the classifications for each of the individuals of the first set of individuals, a readiness risk scoring model that determines a risk that an individual is not ready from health data of the individual, obtaining health data regarding a particular individual, and determining, from the health data regarding the particular individual and the readiness risk scoring model, a risk that the particular individual is not ready.

Other versions of the subject matter include corresponding systems, apparatus, and computer programs, configured to perform the actions of the methods, encoded on computer storage devices or non-storage devices.

These and other versions may each optionally include one or more of the following features. For instance, in some implementations the health data includes a set of health characteristics for each individual of the set of individuals. In some implementations, generating, from the classifications for each of the individuals of the first set of individuals, a readiness risk scoring model that determines a risk that an individual is not ready from health data of the individual includes determining a weight that each characteristic of individuals of the set of individuals has on the classification for the individuals of the set of individuals. In some aspects, the set of health characteristics includes two or more of age, gender, alcohol use, body mass index, and hours of sleep per day.

In certain aspects, determining, from the health data regarding the particular individual and the readiness risk scoring model, a risk that the particular individual is not ready includes determining a readiness risk subscore for each of the health characteristics of the set of health characteristics and aggregating the readiness risk sub-scores for each of the health characteristics of the set of health characteristics into a risk score that indicates the risk that the particular individual is not ready. In some implementations, providing the health data for the set of individuals to a readiness classifier that classifies individuals as either ready or as not ready includes determining whether (i) health data for an individual of the set of individuals before a deployment satisfied health policy requirements and (ii) the individual of the set of individuals successfully completed the deployment and in response to determining that (i) health data for an individual of the set of individuals before the deployment satisfied health policy requirements and (ii) the individual of the set of individuals successfully completed the deployment, classifying the individual of the set of individuals as ready.

In some aspects, providing the health data for the set of individuals to a readiness classifier that classifies individuals as either ready or as not ready includes determining whether (i) health data for an individual of the set of individuals before a deployment satisfied health policy requirements and (ii) the individual of the set of individuals successfully completed the deployment and in response to determining that (i) health data for an individual of the set of individuals before the deployment did not health policy requirements or (ii) the individual of the set of individuals did not successfully complete the deployment, classifying the individual of the set of individuals as not ready. In certain aspects, actions include determining an action that is likely to reduce the risk that the particular individual is not ready and providing for output a suggestion that the action be performed.

In some implementations, determining that the risk that the particular individual is not ready satisfies a warning threshold and in response to determining that the risk that the particular individual is not ready satisfies a warning threshold, providing a warning regarding the risk that the particular individual is not ready. In certain aspects, the health data includes one or more of medical records, biometric data from wearable sensors, and deployment history data.

The details of one or more aspects of the subject matter described in this specification are set forth in the accompanying drawings and the description below. Other features, aspects, and advantages of the subject matter will become apparent from the description, the drawings, and the claims.

### DESCRIPTION OF DRAWINGS

FIG. 1 illustrates a block diagram of a system that determines a likelihood that an individual is ready for deployment.

FIG. 2 is a flow diagram that illustrates an example of a process for determining a likelihood that an individual is ready for deployment.

FIG. 3 is an example of a graphical user interface that indicates a likelihood that an individual is ready for deployment.

FIG. 4 illustrates a schematic diagram of an example computer system.

Like reference symbols in the various drawings indicate like elements.

### DETAILED DESCRIPTION

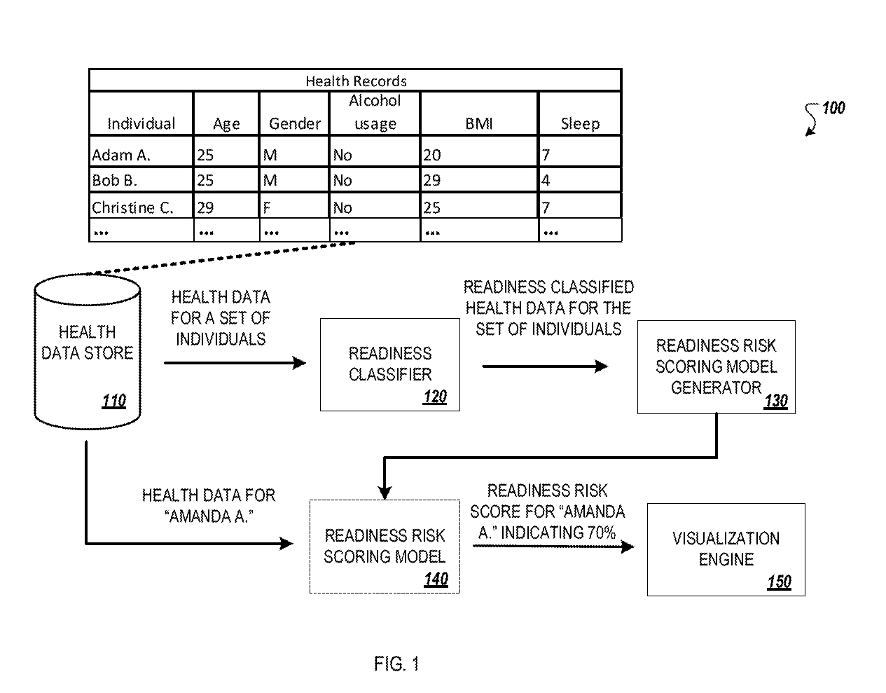

FIG. 1 illustrates a block diagram of a system 100 that determines a likelihood that an individual is ready for deployment. Briefly, and as described further in detail below, the system 100 includes a health data store 110 that stores health data for individuals, a readiness classifier 120 that classifies the individuals as either ready or not ready, a readiness risk scoring model generator 130 to generates a readiness risk scoring model 140, where the readiness risk scoring model 140 may be used to determine a likelihood that a particular individual is ready for deployment, and a visualization engine 150.

The health data store 110 may be one or more databases that store health data regarding a set of individuals. For example, the heath database may store multiple records for thousands of people, where each record indicates health data for a respective individual. The health data may include values for health characteristics that may impact an individual's readiness for deployment. For example, health data may include values for age, gender, alcohol usage, BMI, amount of sleep, deployments, and other characteristics for each individual.

The health data store 110 may store health data that is historical and tracks a period of time. For example, the health data store 110 may store records of an average amount of sleep per day, where a value is stored for each week across a five year history. In another example, health data store 110 may store deployment data that specifies each time an individual was deployed, a duration of each deployment, and, for each deployment, whether the deployment was not completed for health reasons.

The health data store 110 may obtain the health data from a variety of different sources. For example, the health data store 110 may collect the health data from government data sources, public data sources regarding an age of individuals, electronic health records that store test results entered by medical personnel examining the individuals, and wearable biometric devices worn by individuals. The health data store 110 may format the health data obtained from the different sources into a common structured. For example, the health data store 110 may receive unstructured, semi-structured, and structured data from different sources for the same individual and then generate a single record in a structured format shared by all records stored by the health data store 110.

The readiness classifier 120 may receive health data for a set of individuals and classify the individuals as either ready or not ready for deployment based on the health data. For example, the readiness classifier 120 may obtain health data for the individuals “Adam A,” “Bob C,” and “Christine C.” and classify “Adam A.” as ready, classify “Bob B.” as not ready, and classify “Christine C.” as ready.

The readiness classifier 120 may classify individuals as either ready or not ready based on whether the obtained health data for the individuals satisfy health policy requirements. For example, the readiness classifier 120 may obtain pre-determined health policy requirements that specify individuals must have a BMI lower than thirty to be considered ready, or must have at least three hours of sleep to be considered ready. The readiness classifier 120 may determine a health policy requirement for various health characteristics and determine whether the individual satisfies each of the health policy requirements for the various health characteristics. For example, the readiness classifier 120 may determine whether an individual has a BMI less than thirty, has an average amount of sleep greater than three hours a night, is young than fifty and, in response to determining that each of the requirements are satisfied, determine that the individual is ready.

The readiness classifier 120 may additionally classify individuals as either ready or not ready based on whether the individuals successfully completed a deployment. For example, the readiness classifier 120 may classify individuals as not having been ready if the individuals did not successfully complete a deployment for health reasons even if health policy requirements were satisfied by the individuals. In another example, the readiness classifier 120 may classify individuals as having been ready if the individuals did successfully complete a deployment or did not successfully complete a deployment for some other reason other than health reason.

The readiness risk scoring model generator 130 may obtain health data from the readiness classifier 120 in which individuals are classified as either ready or not ready. For example, the readiness risk scoring model generator 130 may obtain, from the readiness classifier 120, health data for “Adam A,” “Bob C,” and “Christine C.” and indications that “Adam A.” was classified as ready, “Bob B.” was classified as not ready, and “Christine C.” was classified as ready.

The generator 130 may generate a readiness risk scoring model from the health data and the classifications. For example, the generator 130 may generate a readiness risk scoring model that receives health data for an individual as input and that, as output, provides a risk score that indicates a likelihood that the individual is not ready. The generator 130 may generate the readiness risk scoring model based on determining a relationship between the health characteristics of individuals and the classifications on whether the individuals are ready. For example, the generator 130 may determine the BMI characteristic has a weight of 20% and the amount of sleep characteristic has a weight of 15% in the classification of whether individuals are ready.

The generator 130 may generate the readiness risk scoring model by using the weight of evidence (WOE) technique. For example, the generator may partition the readiness classified health data into training, validation, and test (if data amount permits) datasets as part of future model validation. The weight of evidence for each health characteristic may be calculated by taking the natural log(In) of the ratios of the percent of individuals classified as ready divided by the percent of individuals classified as not ready. The calculation for weight of evidence may be WoE=ln (% of “Ready”/% of “Not Ready”)

WoE may then be used to bin or group health characteristics. Each bin may include a minimum of five percent of not ready individuals for ensuring a more accurate prediction. One example of an application of the WoE technique for continuous and categorical characteristics is summarized below. For continuous characteristics, five to twenty bins may be created. Bins with similar WoE values may be then combined, and categories may be replaced with WoE values. For categorical characteristics, categories having similar WoE values may be merged forming new characteristic categories. Next, the predictive power of each characteristic in differentiating between ready and not ready may be analyzed by calculating an Information Value (IV) statistic. Each characteristic may then be ranked based on their level of importance to the target. Characteristics having a low, e.g., below 0.3, or high, e.g., greater than 0.5, IV may be considered for removal from the dataset. The formula for Information Value (IV) statistic may be IV=Σ(% of “Ready”−% of “Not Ready”)]·WoE.

The generator 130 may then use a classifier to attempt to fit a model on the training dataset that best describes the relationship between the classification and target characteristics. Multiple candidate models may be developed as part of the model fit process. The equation for the classifier may be ln(p-hat(1-p-hat)=b₀+b₁X₁+b₂X₂+ . . . +bₚXₚ. The resulting classifying models may be compared based on performance accuracy using the validation dataset. Measures of accuracy for identifying a “champion” model may include the misclassification error rate, receiver operating characteristic (ROC), and area under the curve (AUC). A “champion” model based on accuracy may be then selected. The output from the classifier technique may then be used to derive a risk score that reflects a likelihood that an individual is not ready. Calculating the probability may include converting log odds to odds given p, an observed proportion or probability, where odds=p/(1−p), and converting odds to probability, where probability=odds/1+odds.

The readiness risk scoring model 140 generated by the readiness risk scoring model generator 130 may then obtain heath data of a particular individual from the health data store 110 and determine a risk that the particular individual is not ready. For example, the readiness risk scoring model 140 may obtain a health data record for “Amanda A.,” provide that record as input to the readiness risk scoring model 140, and receive an output of a readiness risk score of 70% that indicates that there is a 70% chance that “Amanda A.” is not ready. In this example, “Amanda A.” may satisfy health policy requirements, which is why her readiness risk score is below 100%, but there is a 70% confidence that she actually is not ready.

In some implementations, the readiness risk scoring model 140 may determine a readiness risk subscore for each health characteristic of the health data. For example, a readiness risk scoring model 140 may assign a subscore of fifty for age for an individual twenty five or younger, a subscore of five for gender for a female, a subscore of zero for alcohol usage if the individual doesn't engage in hazardous alcohol usage, a subscore of one hundred seventy five for an individual that has a BMI greater than twenty six, and a subscore of two hundred and five for an individual that has less than four hours of sleep a day, for a total risk score of four hundred thirty five out of a maximum of five hundred. Accordingly, the likelihood that the individual is not ready may be 87%, e.g., four hundred thirty five divided by five hundred.

The visualization engine 150 may provide one or more visualizations to a user of the system by using the determined risk that a particular individual is not ready. For example, the visualization engine 150 may display a risk score that indicates a likelihood that an individual is ready to that individual so that the individual can see the determined score and the individual sub-scores for each of the health characteristics. In some implementations, the visualization engine 150 may display the risk scores for multiple individuals in a group to a leader of a group. For example, the visualization engine 150 may order individuals by decreasing risk and display a list naming the individuals along with showing their respective risk scores in a single graphical user interface. The leader may then contact the individuals near the top of the list to help the individuals reduce their risk scores.

In some implementations, the visualization engine 150 may further indicate a percentage of individuals in a group that are below a risk score threshold. For example, the visualization engine 150 may use a risk score threshold of 75% and indicate a percentage of a group that has a determined risk score of less than or equal to 75%.

In some implementations, the visualization engine 150 may provide suggestions that may result in reduction in a risk that an individual is not ready. For example, the visualization engine 150 may determine that a user's health characteristic of sleep contributed the most to the risk score, e.g., contributed two hundred and five points out of a total of four hundred and thirty five points and a maximum of five hundred, and, in response, determine to provide a suggestion that the user increase their amount of sleep. The suggestion may include displaying in a graphical user interface, “Sleeping less than 6 hours a night? Improve your sleep hygiene by: Ensuring adequate exposure to natural light. Avoiding stimulants such as caffeine and nicotine close to bedtime. Limiting daytime naps to 30 minutes.” In some implementations, the visualization engine 150 may also provide a suggestion for a group of individuals to a leader of that group. For example, the visualization engine 150 may determine that many individuals in a group are engaging in hazardous alcohol usage and display a suggestion to the leader that the group have additional training on healthy alcohol usage.

In some implementation, similarly to how the readiness risk scoring model generator 130 may determine a relationship between health data and classifications of ready and not ready, the system 100 may identify health events that have occurred from the obtained health data and then determine a likelihood that individuals will experience the health events in the future. For example, the system 100 may identify users that engaged in hazardous alcohol usage, determine a correlation between the health characteristics and the hazardous alcohol usage, and then determine a likelihood that other users will engage in hazardous alcohol usage in the future.

Different configurations of the system 100 may be used where functionality of the health data store 110, the readiness classifier 120, the readiness risk scoring model generator 130, the readiness risk scoring model 140, and the visualization engine 150, may be combined, further separated, distributed, or interchanged. For example, the readiness risk scoring model generator 130 may instead classify individuals as ready or not ready.

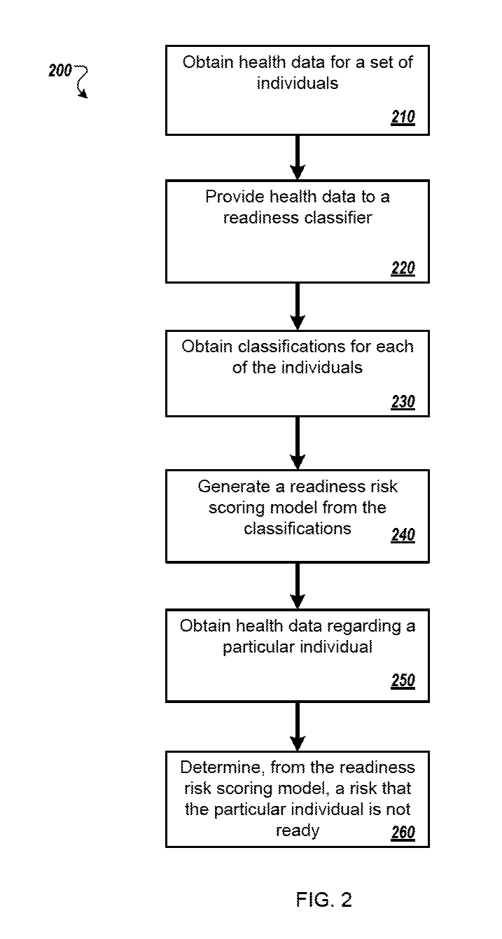

FIG. 2 is a flow diagram that illustrates an example of a process 200 for determining a likelihood that an individual is ready for deployment. The operations of the process 200 may be performed by one or more computing systems, such as the system 100 of FIG. 1.

The process 200 includes obtaining health data for a set of individuals (210). For example, the health data store 110 may obtain values indicating age, gender, alcohol usage, BMI, sleep for each of multiple different individuals from various different sources including governmental sources, electronic medical record data stores, and wearable biometric devices.

The process 200 includes providing health data to a readiness classifier (220). For example, the health data store 110 may provide health data for a set of ten thousand individuals to the readiness classifier 120.

The process 200 includes obtaining classifications for each of the individuals (230). For example, the readiness classifier 120 may receive health data for each individual as input and provide as output a classification as to whether the individual was ready or not ready based on the health data for that individual, without considering health data obtained for other individuals.

The process 200 includes generating a readiness risk scoring model from the classifications (240). For example, the generator 130 may obtain the health data for a set of individuals and corresponding classifications made by the readiness classifier 120, and generate a readiness risk scoring model from the health data and the classifications. As described above in connection with FIG. 1, the generator 130 may use a weight of evidence technique and a classifier technique to generate the model.

The process 200 includes obtaining health data regarding a particular individual (250). For example, the health data store 110 may obtain health data for a particular individual “Amanda A” from a variety of sources. In some implementations, the particular individual and health data may not have already been included in the health data used to generate the readiness risk scoring model from the classifications. In some implementations, the particular individual and health data may have already been included in the health data used to generate the readiness risk scoring model from the classifications.

The process 200 includes determining, from the readiness risk scoring model, a risk that the particular individual is not ready (260). For example, the health data for the particular individual “Amanda A” may be provided as input to the readiness risk scoring model 140 and a readiness risk score that indicates a risk that the particular individual “Amanda A” is not ready may be received as an output from the model 140.

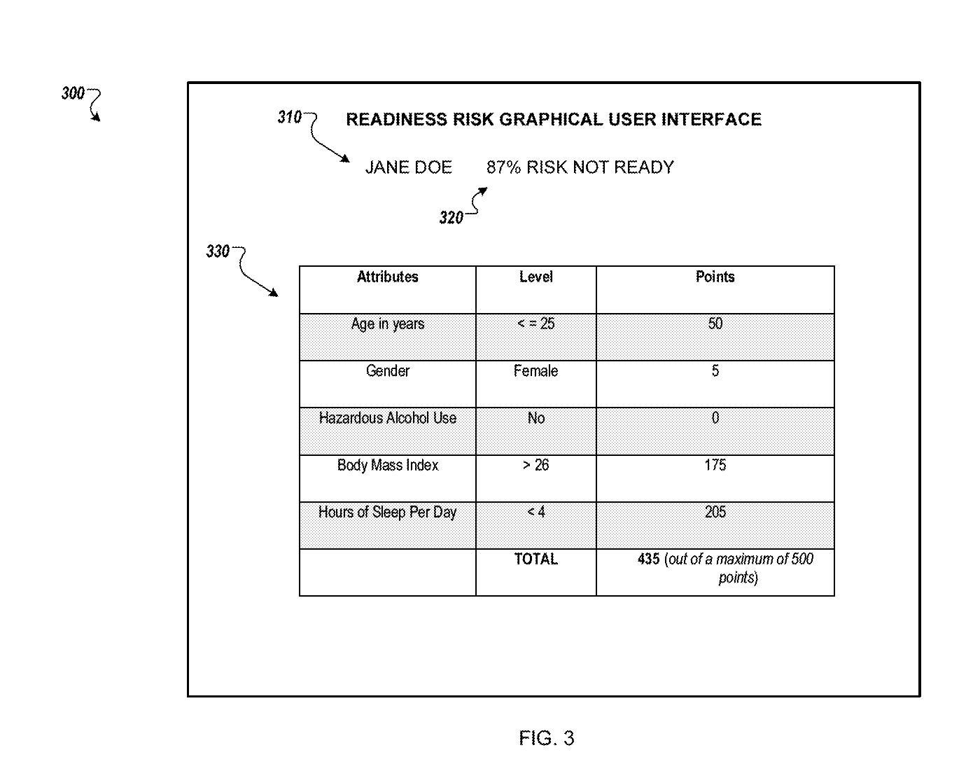

FIG. 3 is an example of a graphical user interface 300 that indicates a likelihood that an individual is ready for deployment. The graphical user interface 300 may include an identifier 310 of an individual, an indication 320 of a likelihood that an individual is ready for deployment, and an indication of sub-scores 330 indicating a contribution of various health characteristics to the likelihood that the individual is ready for deployment. For example, the identifier 310 of an individual may be text displaying a name of “Jane Doe,” the indication 320 of the likelihood that the individual is ready for deployment may be displayed text of “87%” risk that Jane Doe is not ready for deployment, and the indication of sub-scores 330 indicating the contribution of various health characteristics to the likelihood that the individual is ready for deployment may be a displayed table showing, for each health characteristic, a level for each health characteristic that has been assigned to Jane Doe and a risk subscore for Jane Doe corresponding to the level that has been assigned.

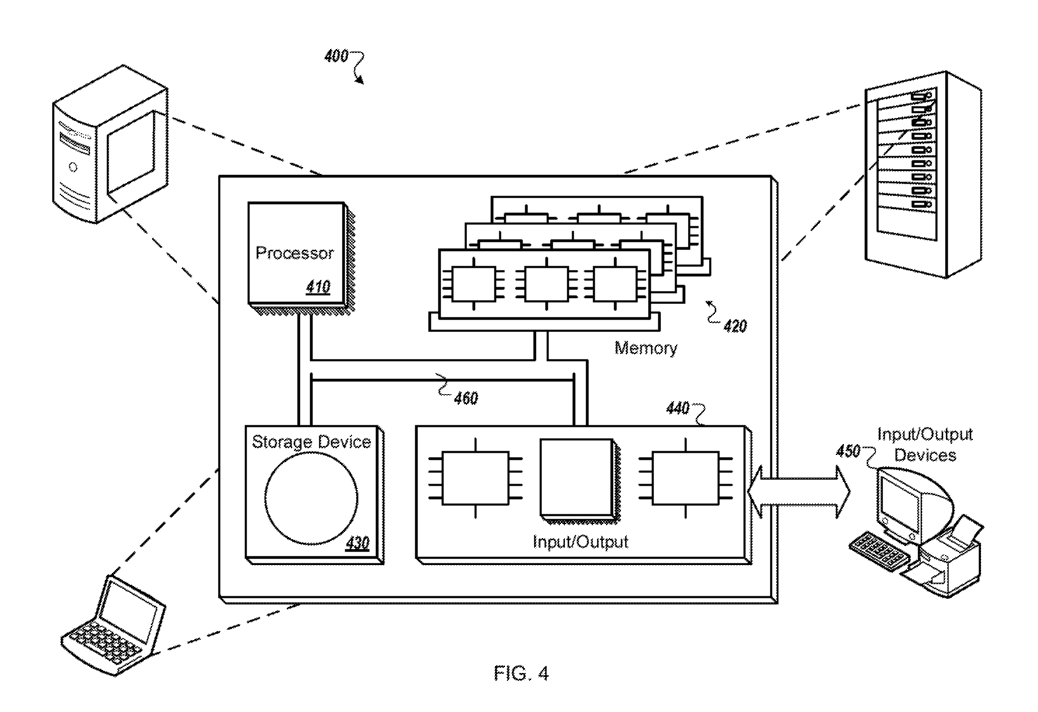

FIG. 4 depicts an example computing system, according to implementations of the present disclosure. The system 400 may be used for any of the operations described with respect to the various implementations discussed herein. For example, the system 400 may be included, at least in part, in one or more of the system 100. The system 400 may include one or more processors 410, a memory 420, one or more storage devices 430, and one or more input/output (I/O) devices 450 controllable through one or more I/O interfaces 440. The various components 410, 420, 430, 440, or 450 may be interconnected through at least one system bus 460, which may enable the transfer of data between the various modules and components of the system 400.

The processor(s) 410 may be configured to process instructions for execution within the system 400. The processor(s) 410 may include single-threaded processor(s), multi-threaded processor(s), or both. The processor(s) 410 may be configured to process instructions stored in the memory 420 or on the storage device(s) 430. The processor(s) 410 may include hardware-based processor(s) each including one or more cores. The processor(s) 410 may include general purpose processor(s), special purpose processor(s), or both.

The memory 420 may store information within the system 400. In some implementations, the memory 420 includes one or more computer-readable media. The memory 420 may include any number of volatile memory units, any number of non-volatile memory units, or both volatile and non-volatile memory units. The memory 420 may include read-only memory, random access memory, or both. In some examples, the memory 420 may be employed as active or physical memory by one or more executing software modules.

The storage device(s) 430 may be configured to provide (e.g., persistent) mass storage for the system 400. In some implementations, the storage device(s) 430 may include one or more computer-readable media. For example, the storage device(s) 430 may include a floppy disk device, a hard disk device, an optical disk device, or a tape device. The storage device(s) 430 may include read-only memory, random access memory, or both. The storage device(s) 430 may include one or more of an internal hard drive, an external hard drive, or a removable drive.

One or both of the memory 420 or the storage device(s) 430 may include one or more computer-readable storage media (CRSM). The CRSM may include one or more of an electronic storage medium, a magnetic storage medium, an optical storage medium, a magneto-optical storage medium, a quantum storage medium, a mechanical computer storage medium, and so forth. The CRSM may provide storage of computer-readable instructions describing data structures, processes, applications, programs, other modules, or other data for the operation of the system 400. In some implementations, the CRSM may include a data store that provides storage of computer-readable instructions or other information in a non-transitory format. The CRSM may be incorporated into the system 400 or may be external with respect to the system 400. The CRSM may include read-only memory, random access memory, or both. One or more CRSM suitable for tangibly embodying computer program instructions and data may include any type of non-volatile memory, including but not limited to: semiconductor memory devices, such as EPROM, EEPROM, and flash memory devices; magnetic disks such as internal hard disks and removable disks; magneto-optical disks; and CD-ROM and DVD-ROM disks. In some examples, the processor(s) 410 and the memory 420 may be supplemented by, or incorporated into, one or more application-specific integrated circuits (ASICs).

The system 400 may include one or more I/O devices 450. The 1/O device(s) 450 may include one or more input devices such as a keyboard, a mouse, a pen, a game controller, a touch input device, an audio input device (e.g., a microphone), a gestural input device, a haptic input device, an image or video capture device (e.g., a camera), or other devices. In some examples, the 1/O device(s) 450 may also include one or more output devices such as a display, LED(s), an audio output device (e.g., a speaker), a printer, a haptic output device, and so forth. The I/O device(s) 450 may be physically incorporated in one or more computing devices of the system 400, or may be external with respect to one or more computing devices of the system 400.

The system 400 may include one or more I/O interfaces 440 to enable components or modules of the system 400 to control, interface with, or otherwise communicate with the I/O device(s) 450. The I/O interface(s) 440 may enable information to be transferred in or out of the system 400, or between components of the system 400, through serial communication, parallel communication, or other types of communication. For example, the I/O interface(s) 440 may comply with a version of the RS-232 standard for serial ports, or with a version of the IEEE 1284 standard for parallel ports. As another example, the I/O interface(s) 440 may be configured to provide a connection over Universal Serial Bus (USB) or Ethernet. In some examples, the I/O interface(s) 440 may be configured to provide a serial connection that is compliant with a version of the IEEE 1394 standard.

The I/O interface(s) 440 may also include one or more network interfaces that enable communications between computing devices in the system 400, or between the system 400 and other network-connected computing systems. The network interface(s) may include one or more network interface controllers (NICs) or other types of transceiver devices configured to send and receive communications over one or more networks using any network protocol.

Computing devices of the system 400 may communicate with one another, or with other computing devices, using one or more networks. Such networks may include public networks such as the internet, private networks such as an institutional or personal intranet, or any combination of private and public networks. The networks may include any type of wired or wireless network, including but not limited to local area networks (LANs), wide area networks (WANs), wireless WANs (WWANs), wireless LANs (WLANs), mobile communications networks (e.g., 3G, 4G, Edge, etc.), and so forth. In some implementations, the communications between computing devices may be encrypted or otherwise secured. For example, communications may employ one or more public or private cryptographic keys, ciphers, digital certificates, or other credentials supported by a security protocol, such as any version of the Secure Sockets Layer (SSL) or the Transport Layer Security (TLS) protocol.

The system 400 may include any number of computing devices of any type. The computing device(s) may include, but are not limited to: a personal computer, a smartphone, a tablet computer, a wearable computer, an implanted computer, a mobile gaming device, an electronic book reader, an automotive computer, a desktop computer, a laptop computer, a notebook computer, a game console, a home entertainment device, a network computer, a server computer, a mainframe computer, a distributed computing device (e.g., a cloud computing device), a microcomputer, a system on a chip (SoC), a system in a package (SiP), and so forth. Although examples herein may describe computing device(s) as physical device(s), implementations are not so limited. In some examples, a computing device may include one or more of a virtual computing environment, a hypervisor, an emulation, or a virtual machine executing on one or more physical computing devices. In some examples, two or more computing devices may include a duster, cloud, farm, or other grouping of multiple devices that coordinate operations to provide load balancing, failover support, parallel processing capabilities, shared storage resources, shared networking capabilities, or other aspects.

Implementations and all of the functional operations described in this specification may be realized in digital electronic circuitry, or in computer software, firmware, or hardware, including the structures disclosed in this specification and their structural equivalents, or in combinations of one or more of them. Implementations may be realized as one or more computer program products, i.e., one or more modules of computer program instructions encoded on a computer readable medium for execution by, or to control the operation of, data processing apparatus. The computer readable medium may be a machine-readable storage device, a machine-readable storage substrate, a memory device, a composition of matter effecting a machine-readable propagated signal, or a combination of one or more of them. The term “computing system” encompasses all apparatus, devices, and machines for processing data, including by way of example a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) may be written in any appropriate form of programming language, including compiled or interpreted languages, and it may be deployed in any appropriate form, including as a standalone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform functions by operating on input data and generating output. The processes and logic flows may also be performed by, and apparatus may also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any appropriate kind of digital computer. Generally, a processor may receive instructions and data from a read only memory or a random access memory or both. Elements of a computer can include a processor for performing instructions and one or more memory devices for storing instructions and data. Generally, a computer may also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory may be supplemented by, or incorporated in, special purpose logic circuitry.

To provide for interaction with a user, implementations may be realized on a computer having a display device, e.g., a CRT (cathode ray tube) or LCD (liquid crystal display) monitor, for displaying information to the user and a keyboard and a pointing device, e.g., a mouse or a trackball, by which the user may provide input to the computer. Other kinds of devices may be used to provide for interaction with a user as well; for example, feedback provided to the user may be any appropriate form of sensory feedback, e.g., visual feedback, auditory feedback, or tactile feedback; and input from the user may be received in any appropriate form, including acoustic, speech, or tactile input.

Implementations may be realized in a computing system that includes a back end component, e.g., as a data server, or that includes a middleware component, e.g., an application server, or that includes a front end component, e.g., a client computer having a graphical UI or a web browser through which a user may interact with an implementation, or any appropriate combination of one or more such back end, middleware, or front end components. The components of the system may be interconnected by any appropriate form or medium of digital data communication, e.g., a communication network. Examples of communication networks include a local area network (“LAN”) and a wide area network (“WAN”), e.g., the Internet.

The computing system may include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of the disclosure or of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some examples be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

A number of implementations have been described. Nevertheless, it will be understood that various modifications may be made without departing from the spirit and scope of the disclosure. For example, various forms of the flows shown above may be used, with steps re-ordered, added, or removed. Accordingly, other implementations are within the scope of the following claims.

What is claimed is:

1. A computer-implemented method comprising:
obtaining health data for a set of individuals;
providing the health data for the set of individuals to a readiness classifier that classifies individuals as either ready or as not ready;
obtaining, from the readiness classifier, classifications for each of the individuals of the set of individuals;
generating, from the classifications for each of the individuals of the first set of individuals, a readiness risk scoring model that determines a risk that an individual is not ready from health data of the individual;
obtaining health data regarding a particular individual; and
determining, from the health data regarding the particular individual and the readiness risk scoring model, a risk that the particular individual is not ready.

2. The method of claim 1, wherein the health data includes a set of health characteristics for each individual of the set of individuals.

3. The method of claim 2, wherein generating, from the classifications for each of the individuals of the first set of individuals, a readiness risk scoring model that determines a risk that an individual is not ready from health data of the individual comprises:
determining a weight that each characteristic of individuals of the set of individuals has on the classification for the individuals of the set of individuals.

4. The method of claim 2, wherein the set of health characteristics includes two or more of age, gender, alcohol use, body mass index, and hours of sleep per day.

5. The method of claim 2, wherein determining, from the health data regarding the particular individual and the readiness risk scoring model, a risk that the particular individual is not ready comprises:
determining a readiness risk subscore for each of the health characteristics of the set of health characteristics; and
aggregating the readiness risk sub-scores for each of the health characteristics of the set of health characteristics into a risk score that indicates the risk that the particular individual is not ready.

6. The method of claim 1, wherein providing the health data for the set of individuals to a readiness classifier that classifies individuals as either ready or as not ready comprises:
determining whether (i) health data for an individual of the set of individuals before a deployment satisfied health policy requirements and (ii) the individual of the set of individuals successfully completed the deployment; and
in response to determining that (i) health data for an individual of the set of individuals before the deployment satisfied health policy requirements and (ii) the individual of the set of individuals successfully completed the deployment, classifying the individual of the set of individuals as ready.

7. The method of claim 1, wherein providing the health data for the set of individuals to a readiness classifier that classifies individuals as either ready or as not ready comprises:
determining whether (i) health data for an individual of the set of individuals before a deployment satisfied health policy requirements and (ii) the individual of the set of individuals successfully completed the deployment; and
in response to determining that (i) health data for an individual of the set of individuals before the deployment did not health policy requirements or (ii) the individual of the set of individuals did not successfully complete the deployment, classifying the individual of the set of individuals as not ready.

8. The method of claim 1, comprising:
determining an action that is likely to reduce the risk that the particular individual is not ready; and
providing for output a suggestion that the action be performed.

9. The method of claim 1, comprising:
determining that the risk that the particular individual is not ready satisfies a warning threshold; and
in response to determining that the risk that the particular individual is not ready satisfies a warning threshold, providing a warning regarding the risk that the particular individual is not ready.

10. The method of claim 1, wherein the health data includes one or more of medical records, biometric data from wearable sensors, and deployment history data.

11. A system comprising:
one or more computers and one or more storage devices storing instructions that are operable, when executed by the one or more computers, to cause the one or more computers to perform operations comprising:
obtaining health data for a set of individuals;
providing the health data for the set of individuals to a readiness classifier that classifies individuals as either ready or as not ready;
obtaining, from the readiness classifier, classifications for each of the individuals of the set of individuals;
generating, from the classifications for each of the individuals of the first set of individuals, a readiness risk scoring model that determines a risk that an individual is not ready from health data of the individual;
obtaining health data regarding a particular individual; and
determining, from the health data regarding the particular individual and the readiness risk scoring model, a risk that the particular individual is not ready.

12. The system of claim 11, wherein the health data includes a set of health characteristics for each individual of the set of individuals.

13. The system of claim 12, wherein generating, from the classifications for each of the individuals of the first set of individuals, a readiness risk scoring model that determines a risk that an individual is not ready from health data of the individual comprises:
determining a weight that each characteristic of individuals of the set of individuals has on the classification for the individuals of the set of individuals.

14. The system of claim 12, wherein the set of health characteristics includes two or more of age, gender, alcohol use, body mass index, and hours of sleep per day.

15. The system of claim 12, wherein determining, from the health data regarding the particular individual and the readiness risk scoring model, a risk that the particular individual is not ready comprises:
determining a readiness risk subscore for each of the health characteristics of the set of health characteristics; and
aggregating the readiness risk sub-scores for each of the health characteristics of the set of health characteristics into a risk score that indicates the risk that the particular individual is not ready.

16. The system of claim 11, wherein providing the health data for the set of individuals to a readiness classifier that classifies individuals as either ready or as not ready comprises:
determining whether (i) health data for an individual of the set of individuals before a deployment satisfied health policy requirements and (ii) the individual of the set of individuals successfully completed the deployment; and
in response to determining that (i) health data for an individual of the set of individuals before the deployment satisfied health policy requirements and (ii) the individual of the set of individuals successfully completed the deployment, classifying the individual of the set of individuals as ready.

17. The system of claim 11, wherein providing the health data for the set of individuals to a readiness classifier that classifies individuals as either ready or as not ready comprises:
determining whether (i) health data for an individual of the set of individuals before a deployment satisfied health policy requirements and (ii) the individual of the set of individuals successfully completed the deployment; and
in response to determining that (i) health data for an individual of the set of individuals before the deployment did not health policy requirements or (ii) the individual of the set of individuals did not successfully complete the deployment, classifying the individual of the set of individuals as not ready.

18. The system of claim 11, the operations comprising:
determining an action that is likely to reduce the risk that the particular individual is not ready; and
providing for output a suggestion that the action be performed.

19. The system of claim 11, the operations comprising:
determining that the risk that the particular individual is not ready satisfies a warning threshold; and
in response to determining that the risk that the particular individual is not ready satisfies a warning threshold, providing a warning regarding the risk that the particular individual is not ready.

20. A non-transitory computer-readable medium storing software comprising instructions executable by one or more computers which, upon such execution, cause the one or more computers to perform operations comprising:
obtaining health data for a set of individuals;
providing the health data for the set of individuals to a readiness classifier that classifies individuals as either ready or as not ready;
obtaining, from the readiness classifier, classifications for each of the individuals of the set of individuals;
generating, from the classifications for each of the individuals of the first set of individuals, a readiness risk scoring model that determines a risk that an individual is not ready from health data of the individual;
obtaining health data regarding a particular individual; and
determining, from the health data regarding the particular individual and the readiness risk scoring model, a risk that the particular individual is not ready.
